(19) United States
(12) Patent Application Publication
(10) Pub. No.: US 2016/0317214 A1
(43) Pub. Date: Nov. 3, 2016

(54) ELECTROSURGICAL FORCEPS WITH SOFT GRIP

(71) Applicant: Kirwan Surgical Products LLC, Marshfield, MA (US)

(72) Inventors: Lawrence T. Kirwan, JR., Kingston, MA (US); Robert T. Boyd, Kingston, MA (US)

(21) Appl. No.: 14/701,768

(22) Filed: May 1, 2015

(51) Int. Cl. A61B 18/14

(52) U.S. Cl. CPC A61B 18/1442; A61B 2018/00178

(57) ABSTRACT

An electrosurgical forceps is provided with a gripping pad formed of a compliant material in the aperture in a gripping region of a blade member of the forceps. The gripping pad includes a pad member, a retaining member and a connecting pillar that extends through an aperture in the blade member.

### CROSS REFERENCE TO RELATED APPLICATIONS

N/A

### STATEMENT REGARDING FEDERALLY SPONSORED RESEARCH OR DEVELOPMENT

N/A

### BACKGROUND

Electrosurgical forceps that are used in surgical procedures have a pair of resilient blades or arms that are used for grasping and coagulating tissue. The forceps may be monopolar or bipolar. In monopolar forceps, the blades are welded or otherwise joined to form an electrode in electrical communication with an electrical generator. Current flows from the active electrode through the patient's tissue to a dispersive electrode in contact with the patient's skin (which may be at some distance from the forceps) and back to the generator. In bipolar forceps, each blade of the pair comprises an electrode in communication with an electrical generator. Current flows from one blade through the tissue to the other blade. A suitable connector for electrical connection to the generator is provided at the proximal end of the forceps.

In some forceps, the blades are curved so that the tips at the distal are offset from the proximal end. The offset provides a better line of sight for the surgeon. Some forceps also include a serrated region along the body of the blades to provide a finger grip for the surgeon.

### SUMMARY OF THE INVENTION

The invention provides an electrosurgical forceps that includes a soft grip. The soft grip comprises a gripping pad formed from a compliant or resilient material provided in a gripping region of the forceps. The material of the gripping pad provides a non-slip or textured surface that is easier for a physician to grasp, particularly when wearing gloves that have become moist or wet. The gripping pad does not interfere with current flow along the electrode blade. It cannot be easily removed from the forceps blade.

### DESCRIPTION OF THE DRAWINGS

The invention will be more fully understood from the following detailed description taken in conjunction with the accompanying drawings in which:

FIG. 1 is a perspective view of a forceps with an embodiment of a gripping pad;

FIG. 2 is a plan view of a blade of the forceps of FIG. 1 with the gripping removed for clarity;

FIG. 3 is a plan view of the blade of the forceps of FIG. 1 with the gripping pad;

FIG. 4 is a partially sectional side view of the forceps of FIG. 1;

FIG. 5 is an exploded, partial view of the forceps of FIG. 1 illustrating an assembly step;

FIG. 6 is a perspective view of the gripping pad of the embodiment of FIG. 1;

FIG. 7 is a top plan view of the gripping pad of FIG. 6;

FIG. 8 is a bottom plan view of the gripping pad of FIG. 6;

FIG. 9 is a side view of the gripping pad of FIG. 6;

FIG. 10 is a further side view of the gripping pad of FIG. 6;

FIG. 11 is a cross-sectional view along line XI-XI of FIG. 9;

FIG. 12 is a cross-sectional view along line XII-XII of FIG. 9;

FIG. 13 is a top plan view of a further embodiment of a gripping pad;

FIG. 14 is a side view of the gripping pad of FIG. 13; and

FIG. 15 is a cross-sectional view along line XV-XV of FIG. 15.

### DETAILED DESCRIPTION OF THE INVENTION

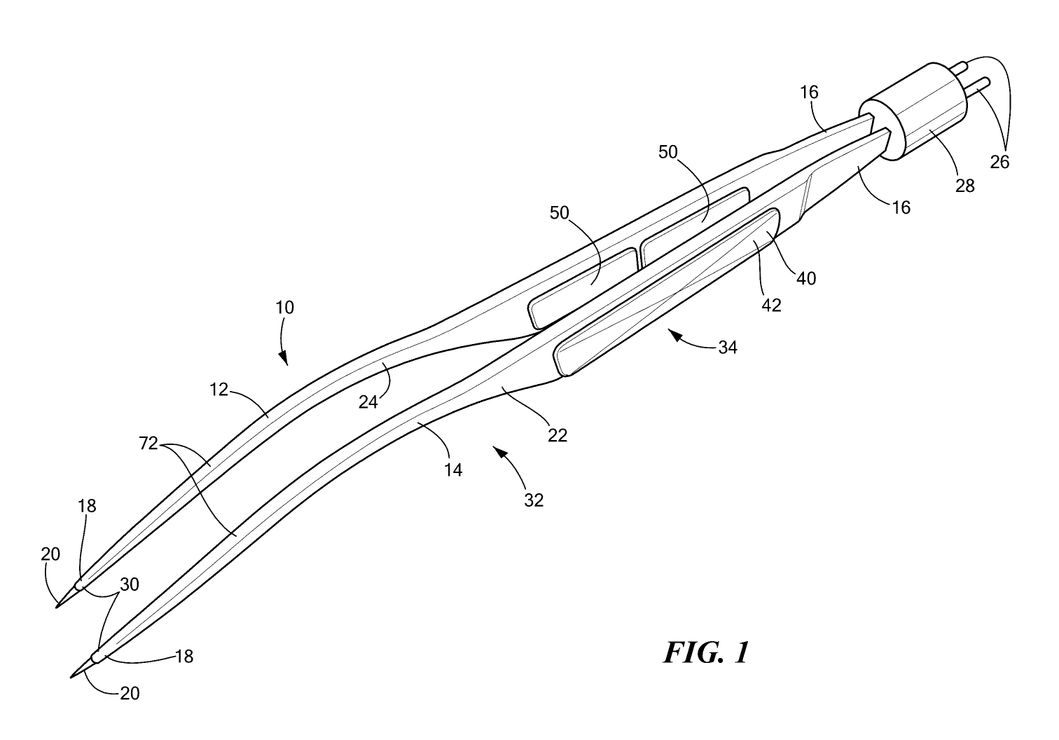

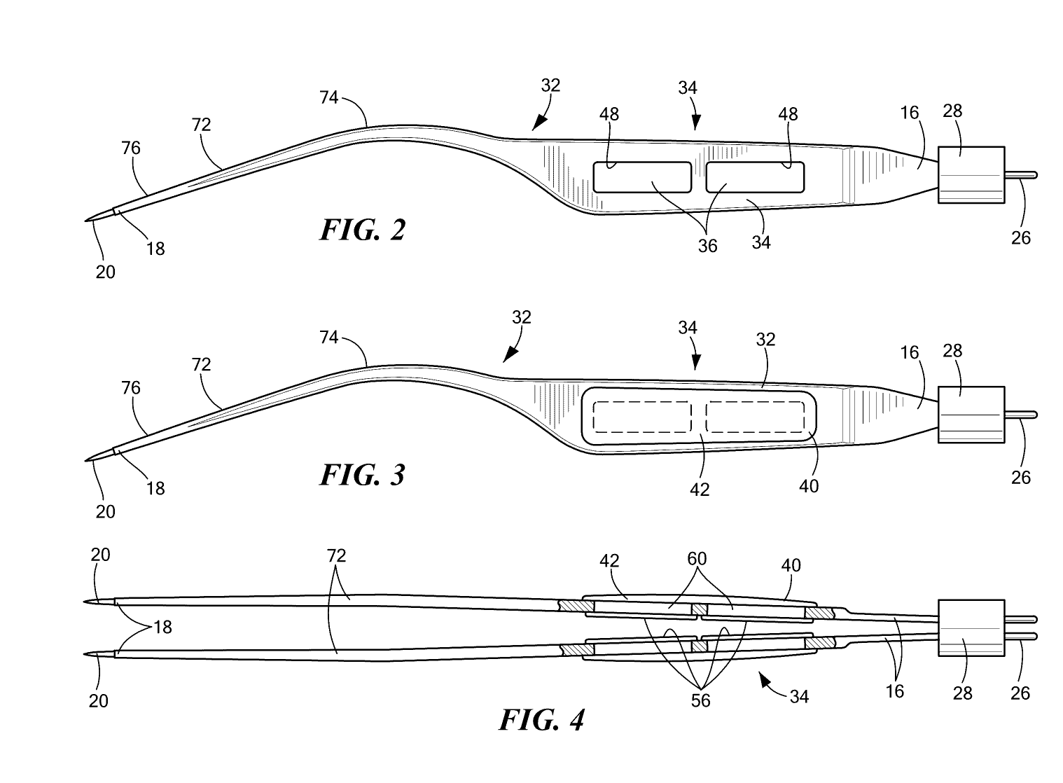

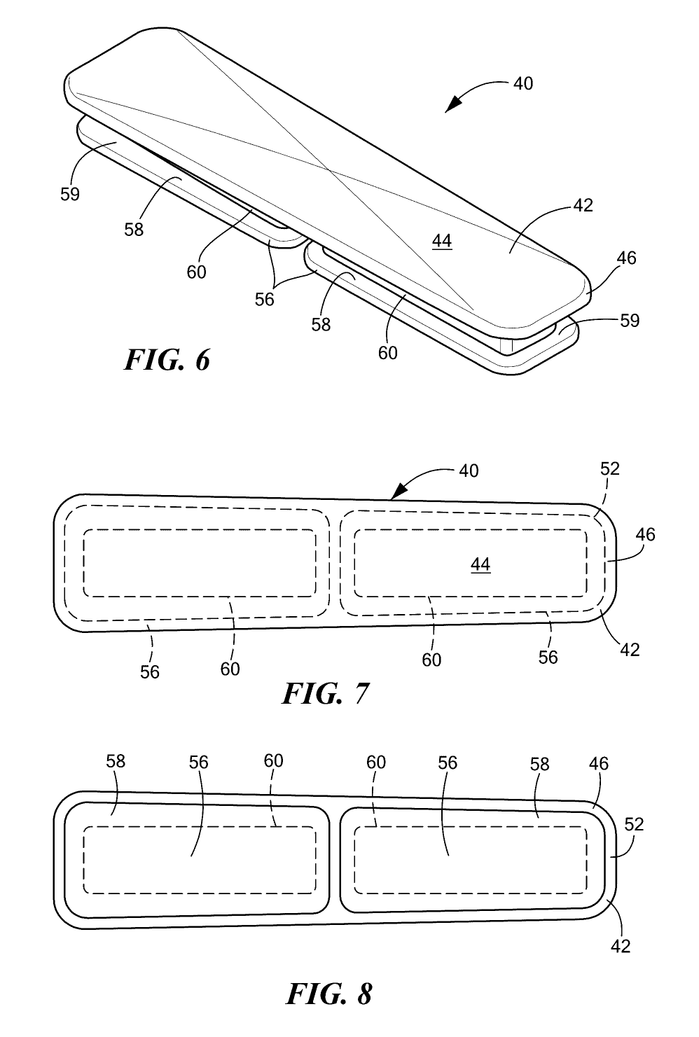

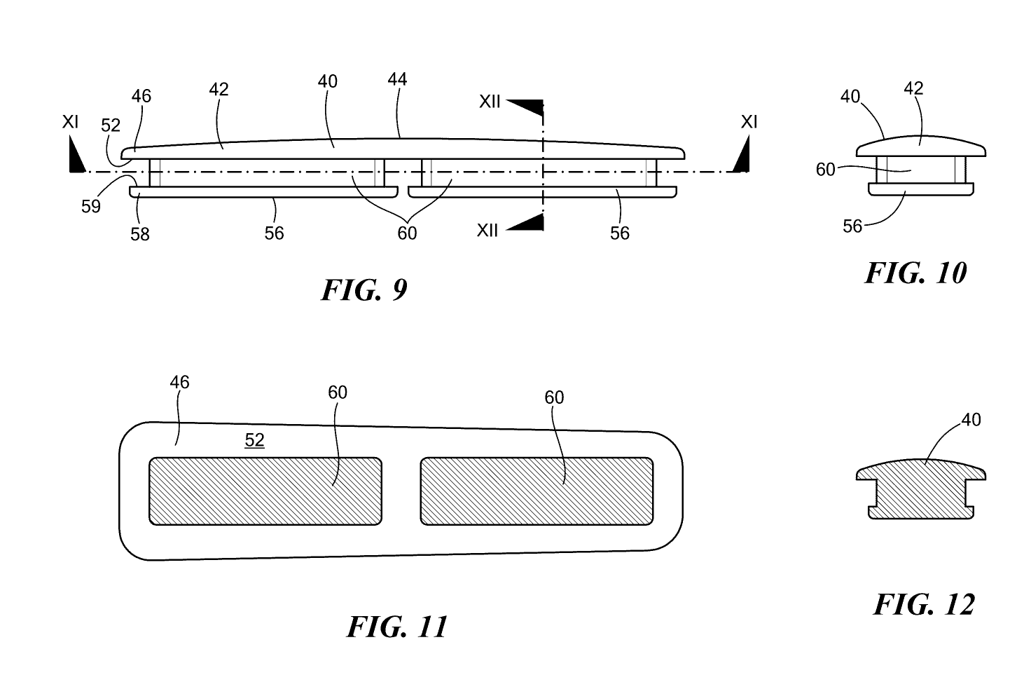

Referring to FIGS. 1-12, a forceps has first and second blade or electrode members 12, 14. Each of the blade members is elongated and extends from a first or proximal end 16 to a second or distal end 18 at a tip 20. The blade members are generally flat, having an outer surface 22 and an inner surface 24, and the tips 20 are configured for gripping tissue between opposed inner surfaces.

The proximal ends 16 are electrically connected in any suitable manner, such as by crimping, welding, or soldering, to terminal pins 26. The proximal ends along with the terminal pins are encapsulated using an epoxy-based material or otherwise mounted within an insulating cap portion 28. The blade members may be insulated with an insulating material 30 along most of their length from the cap portion to a location close to the tip. Alternatively, the blade members may be uninsulated. A plating of an electrically and thermally conductive biocompatible material such as gold may be provided on the tip of an insulated blade member or over the tip and over the entire body of an uninsulated blade member.

The blade members 12, 14 can be formed of any material or combination of materials that provides suitable strength and electrical conductivity. The material may also be thermally conductive in some embodiments. Suitable materials include stainless steel, nickel, copper, silver, titanium, and alloys of any of these metals. Composite materials, such as silver/nickel composites, can also be used.

Each blade member 12, 14 includes a mid portion 32 between the proximal end 16 and the distal end 18. A gripping region 34 is formed in the mid portion. The gripping region includes at least one aperture 36 formed completely through the blade member. In the embodiment shown, two apertures are provided. Current is able to flow around the periphery of the apertures when the forceps are in use.

A gripping pad 40 formed from a compliant or resilient material is disposed in the aperture or apertures 36 in the gripping region 34 of the blade member 12, 14. The gripping pad includes a pad member 42 having a gripping surface 44 for contact by the fingers of the physician when using the forceps. The gripping surface 44 can be flat or curved. In one embodiment, the surface has a slight concave curvature. The pad member 42 has an area that is larger than and overlies the area of the aperture 36, or if multiple apertures 36 are used, a continuous area that is larger than and overlies the multiple apertures. An edge portion 46 of the pad member defines an area that extends beyond the periphery 48 of the aperture or apertures 36. An inner surface 52 of the edge portion 46 is in contact with the outer surface 22 of the blade member 12, 14 continuously around the periphery 48 of the aperture 36 or, if more than one aperture is used, around an outer periphery of the combined apertures 36. The edge portion 46 assists in retaining the gripping pad 40 on the blade 12, 14.

The gripping pad 40 also includes one or more retaining members 56 and one or more connecting pillars 60 between the pad member 42 and the retaining member or members 56. Each connecting pillar 60 extends through an associated aperture 26 in contact with side walls 62 of the aperture, so that it can form a press fit or interference fit within the aperture. Each retaining member includes an edge portion 58 extending beyond the periphery 48 of the adjacent aperture 36 on the inner surface 24 of the blade member 12, 14. An inner surface 59 of the edge portion 58 is in contact with the inner surface 24 of the blade member 12, 14 around the periphery 48 of the aperture 36. The edge portion 58 of the retaining member 56 helps to retain the gripping pad 40 within the aperture 36 on the blade 12, 14. Once the gripping pad 40 has been inserted into the aperture 36 in the blade 12, 14, it is no longer readily removable. Similarly, once installed, the gripping pad does not slide or shift around with respect to the surfaces of the blade. In this manner, the gripping pad provides a stable surface for the physician to grasp the forceps.

In the embodiment shown, two connecting pillars 60 and two retaining members 56 are provided. It will be appreciated that a single connecting pillar 60 and single retaining member 56 can be used. Alternatively, a larger number of connecting pillars and retaining members can be used. Additionally, multiple separated gripping pads 40 can be used. Also, it will be appreciated that a gripping pad or pads can be provided on only one of the two blades of the forceps if desired.

In the embodiment illustrated, the mid portion 32 of each blade 12, 14 includes an elongated arm 72 extending between the gripping region 34 and the distal end 18. Each arm has a width that is more narrow than a width of the gripping region. Also, each arm includes a curved portion 74 extending from the gripping region and narrowing or tapering in width toward the distal end, and a straight portion 76 between the curved portion 74 and the distal end 18. This configuration provides a clear line of sight to the tip 20 for the physician when holding the forceps at the gripping region. It will be appreciated that the blades can also be straight or have other curved configurations.

The gripping pad 40 is formed of a medical grade soft or gel-like material, such as a thermoplastic elastomer (TPE), rubber, silicone rubber, or flexible polyvinyl chloride (PVC). Suitable TPEs include thermoplastic polyurethanes. In one embodiment, the material is selected to have good slip resistance when moist or wet, such as when contacted by a surgeon wearing a wet glove. One suitable thermoplastic elastomer known as C-Flex® is commercially available from Saint-Gobain. In some embodiments, the material can have a hardness value less than 50 on the Shore A scale. Materials with other hardness values can be used. Other materials can be used if desired, such as, without limitation, styrenic block copolymers, polyolefin blends, elastomeric alloys, thermoplastic copolyesters, and thermoplastic polyamides; natural and synthetic rubbers such as, nitrile, millable polyurethane, silicone, butyl and neoprene, or other thermoset elastomers or other thermoplastic polymers. A plasticizer is generally not necessary with many suitable materials but could be used if desired, depending on the material and application.

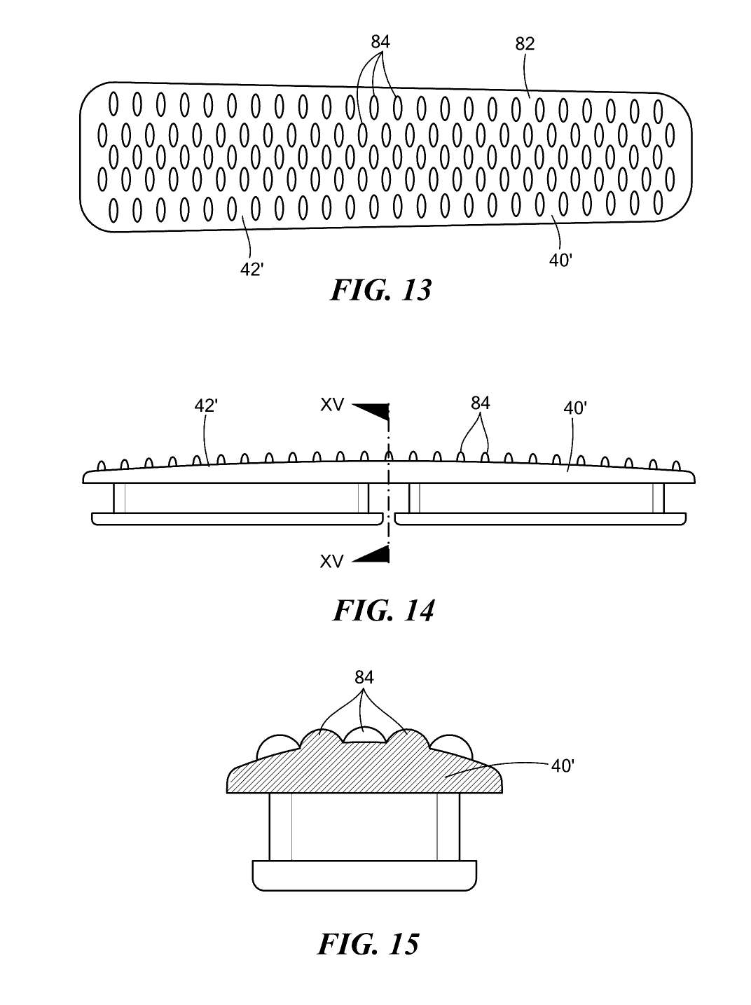

In one embodiment, the gripping surface of the pad member 42′ of gripping pad 40′ can include a texture 82. For example, the surface can include raised or recessed elements. The raised or recessed elements can have any shape or configuration. In the embodiment illustrated in FIGS. 13-15, recessed elements 84 have an oval shape. However, other shapes or configurations, such as, without limitation, diamonds, squares, rectangles, circles, or lines, can be used. The raised or recessed elements can have a variety of sizes, dimensions, or aspect ratios. The elements can be placed in an orderly or regular pattern or can be placed randomly.

The gripping pad can be manufactured in any suitable manner. In one embodiment, the gripping pad is molded, such as by injection molding. In another embodiment, the gripping pad can be overmolded on the blade. In a further embodiment, the gripping pad can be fabricated by an additive manufacturing technique or three-dimensional printing technique.

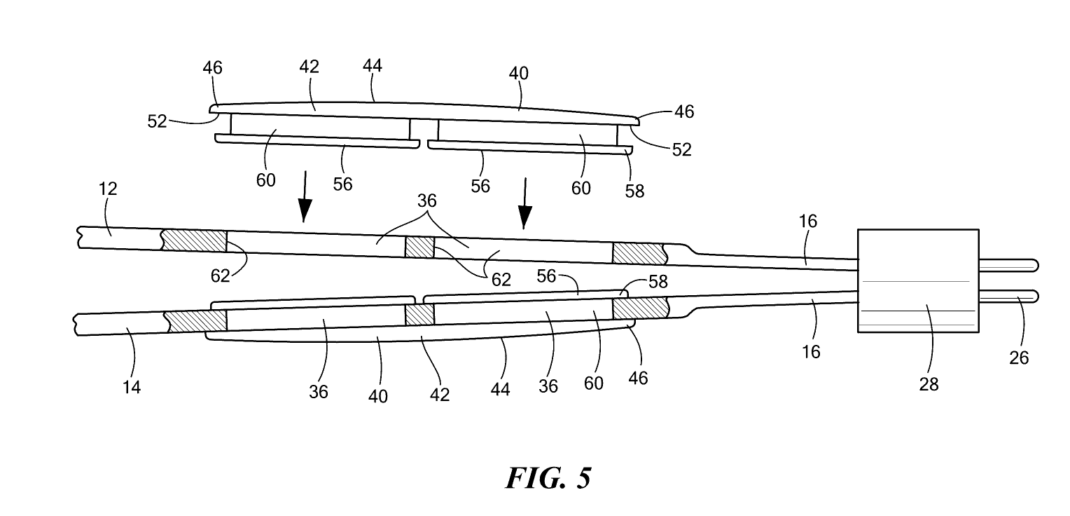

After fabrication, the gripping pad is press fit into the aperture or apertures in the blade. See FIG. 5. The edge portion of the retaining member is sufficiently compliant to deform as it passes through the aperture, and then to return to its original shape after passing through the aperture. Once the gripping pad has been fitted within the aperture, it cannot be removed except with difficulty. The blades are then attached at their proximal ends in any suitable manner to form a complete forceps.

It will be appreciated that the various features of the embodiments described herein can be combined in a variety of ways. For example, a feature described in conjunction with one embodiment may be included in another embodiment even if not explicitly described in conjunction with that embodiment.

The present invention has been described in conjunction with certain preferred embodiments. It is to be understood that the invention is not limited to the exact details of construction, operation, exact materials or embodiments shown and described, and that various modifications, substitutions of equivalents, alterations to the compositions, and other changes to the embodiments disclosed herein will be apparent to one of skill in the art.

What is claimed is:

1. An electrosurgical forceps comprising:
an insulated cap portion;
at least one terminal extending from and fixed to the cap portion;
a pair of blade members, each blade member comprising an outer surface and an inner surface and being generally elongated from a proximal end to a distal end, a tip formed at the distal end, the proximal end fixed within the cap portion, and a mid portion between the proximal end and the distal end, the at least one of the pair of blade members electrically connected to the at least one terminal within the cap portion;
the mid portion of at least one blade member comprising a gripping region, at least one aperture formed through the blade member in the gripping region, the aperture having a periphery; and
a gripping pad comprising a compliant material disposed in the aperture in the gripping region of the at least one blade member, the gripping pad comprising a pad member, a retaining member and a connecting pillar between the pad member and the retaining member, wherein:
the connecting pillar extends through the aperture,
the pad member overlies the aperture and includes an edge portion extending beyond the periphery of the aperture on the outer surface of the blade member, an inner surface of the edge portion in contact with the outer surface of the blade member around the periphery of the aperture, and
the retaining member comprises an edge portion extending beyond the periphery of the aperture on the inner surface of the blade member, an inner surface of the edge portion in contact with the inner surface of the blade member around the periphery of the aperture.

2. The forceps of claim 1, wherein the gripping pad comprises a rubber, a silicone rubber, a thermoplastic elastomer material, or a flexible polyvinyl chloride.

3. The forceps of claim 1, wherein the pad member of the gripping pad includes an outer textured surface or an outer non-slip surface.

4. The forceps of claim 1, wherein the connecting pillar is in contact with side walls of the aperture.

5. The forceps of claim 1, further comprising a further aperture formed through the blade member in the gripping region, the further aperture having a periphery; and
wherein the gripping pad comprises a further retaining member and a further connecting pillar between the pad member and the further retaining member.

6. The forceps of claim 1, wherein the mid portion of each blade further comprises an elongated arm extending between the gripping region and the distal end.

7. The forceps of claim 6, wherein each arm has a width that is more narrow than a width of the gripping region.

8. The forceps of claim 1, wherein the mid portion of each blade further comprises a curved portion.
